(19) United States
(12) Patent Application Publication
(10) Pub. No.: US 2025/0049667 A1
(43) Pub. Date: Feb. 13, 2025

(54) SUNSCREEN OR DAILY CARE COMPOSITION COMPRISING BIS-ETHYLHEXYLOXYPHENOL METHOXYPHENYL TRIAZINE

(71) Applicant: BASF SE, Ludwigshafen am Rhein (DE)

(72) Inventors: Myriam SOHN, Grenzach-Wyhlen (DE); Stanislaw KRUS, Grenzach-Wyhlen (DE)

(21) Appl. No.: 18/797,690

(22) Filed: Aug. 8, 2024

Related U.S. Application Data
(60) Provisional application No. 63/531,854, filed on Aug. 10, 2023.

(51) Int. Cl. A61K 8/37; A61Q 17/04

(52) U.S. Cl. CPC A61K 8/37; A61Q 17/04; A61K 2800/26; A61K 2800/30

(57) ABSTRACT

The present invention relates to a sunscreen or daily care composition comprising
(i) bis-ethylhexyloxyphenol methoxyphenyl triazine (bemotrizinol),
(ii) 1-(4-(1,1-dimethylethyl)phenyl)-3-(4-methoxyphenyl)propane-1,3-dione (avobenzone),
(iii) a water-soluble organic UV filter,
(iv) a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, bis(cyano butylacetate) anthracenediylidene, polyester-25, butyloctyl salicylate and combinations thereof.

### CROSS-REFERENCE TO RELATED APPLICATIONS

This application claims benefit of U.S. Application No. 63/531,854, filed Aug. 10, 2023, which is incorporated herein by reference in its entirety.

### TECHNICAL FIELD

The present application relates to a sunscreen or daily care composition comprising (i) bis-ethylhexyloxyphenol methoxyphenyl triazine (bemotrizinol), (ii) 1-(4-(1,1-dimethylethyl)phenyl)-3-(4-methoxyphenyl)propane-1,3-dione (avobenzone), (iii) a water-soluble organic UV filter, and (iv) a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, Bis(Cyano Butylacetate) Anthracenediylidene, polyester-25, butyloctyl salicylate and combinations thereof.

### BACKGROUND

UV radiation causes harmful effects on the human skin. Beside the acute effect of sunburn of the skin, UV radiation is also known to increase the risk of skin cancer. Furthermore, long time exposure to UV-A and UV-B light can cause phototoxic and photo allergenic reactions on the skin and can accelerate skin aging.

To protect the human skin from UV radiation, various sun protecting UV filters (also referred to as UV absorbers) exist including UV-A filter, UV-B filter and broadband filters. These filters are added to sunscreen or daily care compositions. The UV filters are either organic or inorganic, particulate or non-particulate compounds, of which all have a high absorption efficacy in the UV-light range. In general, UV light can be divided into UV-A radiation and UV-B radiation.

Depending on the position of the absorption maxima, UV-filters are divided into UV-A and UV-B filters. In case an UV-filter absorbs both, UV-A and UV-B light, it is referred to as a broadband absorber. Since broadband UV filters provide protection against UVA and UVB radiation it is preferred to add these kind of UV filters into sunscreen or daily care composition to ensure a broad and efficient UV protection foundation over the whole UV range. However, there is still the need of improved formulations that provide efficient and safe UV protection. Particularly efficient UV filter-containing compositions provide a high SPF value whereas particularly safe UV filter-containing compositions comprise as little UV filter as possible in order to minimize the risk of any potential allergic reaction. Further, particularly efficient UV filter-containing compositions also are photostable. UV filter systems should not photodegrade since photounstable UV filter mixtures can produce unknown photodegradation products or free radicals which can lead to adverse effects such as skin irritation and instability of the sunscreen.

Bis-ethylhexyloxyphenol methoxyphenyl triazine (Bemotrizinol, compound (1)) (abbreviation: BEMT) is a broadband UV absorber. BEMT exhibits a wide and high sun protection band in the UV-A and UV-B range with a E1,1 (extinction 1% filter at an optical thickness of 1 cm) of >730 in the UVB and higher than 800 in the UVA range while having a strong photostability and a good health and environmental profile.

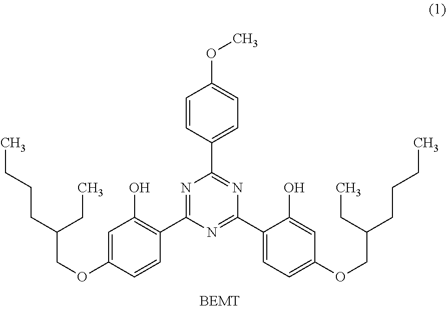 [SMILES: CCCCC(CC)COc1ccc(-c2nc(-c3ccc(OC)cc3)nc(-c3ccc(OCC(CC)CCCC)cc3O)n2)c(O)c1]

Avobenzone (compound (2)) is an effective UV-A absorber.

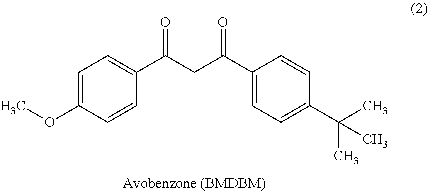 [SMILES: COc1ccc(C(=O)CC(=O)c2ccc(C(C)(C)C)cc2)cc1]

A composition comprising only BEMT and avobenzone as UV filter already provides a decent level of sun protection and can be used for sunscreens with a low to medium sun protection factor. However, further increases in SPF require that additional UV absorbers are provided. In this regard, water-soluble organic UV filters such as ensulizole, ecamsule, and bisdisulizole disodium represent an excellent option since they are not harmful to the human health and are environmentally benign.

Unfortunately, it was discovered that sunscreen compositions comprising (i) bemotrizinol, (ii) avobenzone, and (iii) a water-soluble organic UV filter exhibit low water resistance. This can represent a major obstacle in using such a UV filter combination in sunscreen products because the effectiveness of the sunscreen may already be negatively impacted by sweat or a short stay in water. Since sunscreen products are particularly important in hot weather and during a stay on the beach, a low water resistance represents a major disadvantage for the aforementioned UV filter combination.

Therefore, it was an object of the present invention to provide a sunscreen composition comprising (i) bemotrizinol, (ii) avobenzone, and (iii) a water-soluble organic UV filter which exhibits an increased water-resistance.

### SUMMARY OF THE INVENTION

Surprisingly, the water resistance of a sunscreen or daily care composition comprising (i) bemotrizinol, (ii) avobenzone, and (iii) a water-soluble organic UV filter can be increased significantly by adding a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, Bis(Cyano Butylacetate) Anthracenediylidene polyester-25, butyloctyl salicylate and combinations thereof. It was surprisingly discovered that this class of compounds increases the water resistance of a composition comprising (i) bemotrizinol, (ii) avobenzone, and (iii) a water-soluble organic UV filter to a water resistance of more than 50%.

Therefore, the present invention relates in a first aspect to a sunscreen or daily care composition comprising
(i) bis-ethylhexyloxyphenol methoxyphenyl triazine (bemotrizinol),
(ii) 1-(4-(1,1-dimethylethyl)phenyl)-3-(4-methoxyphenyl)propane-1,3-dione (avobenzone),
(iii) a water-soluble organic UV filter,
(iv) a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, Bis(Cyano Butylacetate) Anthracenediylidene, polyester-25, butyloctyl salicylate and combinations thereof.

In a second aspect, the present invention relates to a method for increasing the water resistance of a sunscreen or daily care composition comprising the step of adding a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, Bis(Cyano Butylacetate) Anthracenediylidene, polyester-25, butyloctyl salicylate and combinations thereof to a sunscreen or daily care composition comprising (i) bis-ethylhexyloxyphenol methoxyphenyl triazine (bemotrizinol), (ii) 1-(4-(1,1-dimethylethyl)phenyl)-3-(4-methoxyphenyl)propane-1,3-dione (avobenzone), and (iii) a water-soluble organic UV filter.

The present invention will be described with respect to particular embodiments and with reference to certain examples, but the invention is not limited thereto, and it is only defined by the appending claims.

### Definitions

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20% preferably ±15%, more preferably ±10%, and even more preferably ±5%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only and is not intended to limit the scope of the present invention that will be limited only by the appended claims. Unless defined otherwise, all technical and scientific terms used herein have the same meanings as commonly understood by one of ordinary skill in the art.

As used herein the term “does not comprise” or “free of” means in the context that the composition of the present invention is free of a specific compound or group of compounds, which may be combined under a collective term, that the composition does not comprise said compound or group of compounds in an amount of more than 0.10% by weight, based on the total weight of the composition. Furthermore, it is preferred that the composition according to the present invention does not comprise said compounds or group of compounds in an amount of more than 0.05% by weight, preferably the composition does not comprise said compounds or group of compounds at all.

When referring to compositions and the weight percent of the therein comprised ingredients it is to be understood that according to the present invention the overall amount of ingredients does not exceed 100% (±1% due to rounding).

The term “cosmetic composition” refers to any topical product which is intended to be applied to the human body for cleansing, beautifying, promoting attractiveness, altering the appearance, or protecting the skin without affecting the body's structure or function. Possible cosmetic compositions within the meaning of the invention comprise leave-on and rinse-off products such as face and body care including daily care compositions with or without UV protection and sunscreen or sunscreen compositions, hair care products such as shampoos, conditioners, shower gels, make-up compositions and similar compositions.

The term “sunscreen composition” or “sunscreen” refers to any topical product which absorbs, and which may further reflect and scatter certain parts of UV radiation. Thus, the term “sunscreen composition” is to be understood as not only including sunscreen compositions, but also any cosmetic compositions that provide UV protection. The term “topical product” refers to a product that is applied to the skin and can refer, e.g., to sprays, lotions, creams, oils, foams, powders, or gels. According to the present invention the sunscreen composition may comprise one or more active agents, e.g., organic and inorganic UV filters, as well as other ingredients or additives, e.g., emulsifiers, emollients, viscosity regulators, stabilizers, preservatives, or fragrances.

The term “daily care composition” refers to any topical product which is intended to be used daily without or with a UV protection. In case UV protection is included, it absorbs and may further reflect and scatter certain parts of UV radiation and is used as an everyday care product for the human body, e.g. for face or body. The daily care composition may comprise one or more active agents, e.g., organic and/or inorganic UV filters, as well as other ingredients or additives, e.g., emulsifiers, emollients, viscosity regulators, stabilizers, preservatives, or fragrances. Suitable daily care composition are according to the present invention, e.g. leave-on face and body care products.

Suitable leave-on products for face and body are, e.g. sunscreen preparations and skin care preparations.

Suitable skin care preparations are e.g., moisturizing, anti-ageing, refining, and lifting preparations. The cited leave-on compositions can be in the form of creams, ointments, pastes, foams, gels, lotions, powders, make-ups, sprays, sticks or aerosols. Daily-care compositions with UV protection protect against damages on human skin by UV light.

The term “UV filter” or “ultraviolet filter” as used herein refers to organic or inorganic compounds, which can absorb and may further reflect and scatter UV radiation caused by sunlight. UV-filter can be classified based on their UV absorbance profile as UV-A, UV-B, or broadband filters. Preferably, the term “UV filter” comprises or consists of any UV filter as defined in the Annex VI (version of 11.11.2022) of the Regulation (EC) No 1223/2009 of the European Parliament and of the Council.

Water soluble UV filters have a solubility in water of at least 2% by weight, preferably at least 3% by weight, more preferably at least 5% by weight.

Oil soluble filters have a solubility in specific emollients of at least 2% by weight, preferably at least 3% by weight, more preferably at least 5% by weight.

The term “emollient” relates to cosmetic specific oils used for protecting, moisturizing, and lubricating the skin. The word emollient is derived from the Latin word mollire, to soften. In general, emollients prevent evaporation of water from the skin by forming an occlusive coating. Alternatively, or additionally, the emollients improve the distribution of the product on the skin. Emollients can be divided into different groups depending on their chemistry and/or polarity index.

The term “sensitive skin” refers to skin of which the natural barrier function is weakened and has broken due to a trigger. A trigger can be for example cold weather, extremely hot water, or critical ingredients, which may be included in sunscreen or daily care compositions.

The term “sun protection factor (SPF)” as used herein indicates how well the skin is protected by a sunscreen composition mainly from UV-B radiation. In particular, the factor indicates how much longer the protected skin may be exposed to the sun without getting a sunburn in comparison to untreated skin. For example, if a sunscreen composition with an SPF of 15 is evenly applied to the skin of a person usually getting a sunburn after 10 minutes in the sun, the sunscreen allows the skilled person to stay in the sun 15 times longer. In other words, SPF 15 means that 1/15 of the burning UV radiation will reach the skin, assuming sunscreen is applied evenly at a dosage of 2 milligrams per square centimeter (mg/cm2).

The definition of “broadband” protection (also referred to as broad-spectrum or broad protection) may be based for example on the “critical wavelength” or the UVA1/UV ratio or the UVA-PF. For broadband coverage, UV-B and UV-A protection must be provided. According to the US requirements, a critical wavelength of at least 370 nm is required for achieving broad spectrum protection. In the proposed order (OTC000008) published Sep. 24, 2021, the FDA proposed to add an additional requirement to pass the broad-spectrum test, the product shall meet a UVA1/UV ratio of at least 0.7. Furthermore, it is recommended by the European Commission that all sunscreens or cosmetic compositions should have an UV-A protection factor, which is at least one third of the labelled sun protection factor (SPF), e.g. if the sunscreen composition has an SPF of 30 the UVA protection factor has to be at least 10.

The term “critical wavelength” is defined as the wavelength at which the area under the UV protection curve (% protection versus wavelength) represents 90% of the total area under the curve in the UV region (290-400 nm). For example, a critical wavelength of 370 nm indicates that the protection of the sunscreen composition is not limited to the wavelengths of UV-B, i.e. wavelengths from 290-320 nm, but extends to 370 nm in such a way that 90% of the total area under the protective curve in the UV region are reached at minimum at 370 nm.

The term “administration” refers to the application of a sunscreen or daily care composition to the skin of a person.

### Particular Embodiments of the Invention

The first aspect of the present invention relates to a sunscreen or daily care composition comprising
(i) bis-ethylhexyloxyphenol methoxyphenyl triazine (bemotrizinol),
(ii) 1-(4-(1,1-dimethylethyl)phenyl)-3-(4-methoxyphenyl)propane-1,3-dione (avobenzone),
(iii) a water-soluble organic UV filter,
(iv) a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, Bis(Cyano Butylacetate) Anthracenediylidene, polyester-25, butyloctyl salicylate and combinations thereof. Such a sunscreen provides UV protection across the entire harmful UV light range while allowing for an increased water resistance.

According to a preferred embodiment of the present invention, the sunscreen or daily care composition is free of the UV filters from the group consisting of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol](bisoctrizole), 3,3,5-Trimethylcyclohexyl 2-hydroxybenzoate (INCI Homosalate), (RS)-2-Ethylhexyl (2E)-3-(4-methoxyphenyl)prop-2-enoate (INCI ethylhexyl methoxycinnamate), and 2-hydroxy-4-methoxybenzophenone (INCI benzophenone-3). It is generally desirable to limit the number of different UV filter in the sunscreen or daily care composition to reduce the risk of component incompatibilities and allergic reactions. Additionally, at least some of the UV filters from the group have negative health- and/or environmental effects.

According to one embodiment, the sunscreen or daily care composition, the sunscreen or daily care composition comprises ethylhexyl 2-cyano-3,3-diphenylprop-2-enoate (INCI octocrylene). According to another embodiment, the sunscreen or daily care composition is free of ethylhexyl 2-cyano-3,3-diphenylprop-2-enoate (INCI octocrylene).

According to another preferred embodiment, the sunscreen or daily care composition is free of any further organic UV filter. The combination of UV filters (i)-(iii) in the present invention is sufficient to provide UV protection across the entire UV range. Limiting the number of different UV filters present in the composition also reduces the risk for unintended interactions or reactions of these different components. It also reduces the risk for any allergic reactions due to other types of UV filters. The term “organic UV filter” is defined as any organic compound that is a UV filter as listed in the Annex VI (version of 11.11.2022) of the Regulation (EC) No 1223/2009 of the European Parliament and of the Council.

According to another preferred embodiment of the invention, the sunscreen or daily care composition is free of zinc oxide. According to another preferred embodiment of the invention, the sunscreen or daily care composition is free of titanium oxide. According to another preferred embodiment of the invention, the sunscreen or daily care composition is free of zinc oxide and titanium oxide. According to another preferred embodiment, the sunscreen or daily care composition is free of any inorganic UV filter.

The sunscreen or daily care composition of the present invention comprises (i) bemotrizinol and (ii) avobenzone. In general, the weight ratio of these two components can vary broadly. In a preferred embodiment of the invention, the weight ratio of (i) bemotrizinol to (ii) avobenzone is from 1:3 to 3:1. Therefore, the total weight amount of BEMT in the sunscreen or daily care composition in this preferred embodiment is at most three times the total weight amount of avobenzone in the sunscreen or daily care composition and the total weight amount of avobenzone in the composition is at most three times the total weight amount of BEMT. It is also possible that the weight ratio of (i) bis-ethylhexyloxyphenol methoxyphenyl triazine (bemotrizinol) to (ii) 1-(4-(1,1-dimethylethyl)phenyl)-3-(4-methoxyphenyl)propane-1,3-dione (avobenzone) is from 1:2.5 to 2.5:1, from 1:2 to 2:1, from 1:1.5 to 1.5:1 or 1:1.

The inventive sunscreen or daily care composition comprises (iv) a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, Bis(Cyano Butylacetate) Anthracenediylidene, polyester-25, butyloctyl salicylate and combinations. According to a particularly preferred embodiment of the invention, the compound is butyloctyl salicylate. The content of the compound in the sunscreen or daily care composition can vary broadly, however, particularly preferred amounts of compound (iv) are from 0.5 to 20.0 wt. %, 1.0 to 18.0 wt. %, 2.0 to 15.0 wt. %, 3.0 to 12.0 wt. %, 4.0 to 10.0 wt. %, or 5.0 to 8.0 wt. %, or 5.5 to 7.5 wt. %, or 6.0 to 7.0 wt. %, in particular also 4.5, 5.0, 5.5, 6.0, 6.5, 7.0, 7.5, or 8.0 wt. %, each based on the total weight of the sunscreen or daily care composition.

The inventive sunscreen or daily care composition comprises (iii) a water-soluble organic UV filter. It is preferred that the water-soluble organic UV filter (iii) is selected from the group consisting of Phenyl-3H-benzimidazole-5-sulfonic acid (Ensulizole), [(3Z)-3-[[4-[(Z)-[7,7-Dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (Ecamsule), disodium phenyl dibenzimidazole tetrasulfonate (Bisdisulizole disodium), and mixtures thereof. Ensulizole, Ecamsule, and Bisdisulizole disodium are well-established organic, water-soluble UV filters with good UV absorbing properties. According to a more preferred embodiment, the water-soluble organic UV filter (iii) is selected from Ensulizole and Ecamsule. Most preferred, the water-soluble organic UV filter (iii) is Ensulizole. Ensulizole is a UV-B filter with minor UV-A protection.

In one embodiment of the invention, Ensulizole is used as a salt, in particular as a salt of an organic nitrogen compound, in particular as a salt of an amine base compound, in particular as tromethamine Ensulizole, triethanolamine Ensulizole, tetrahydroxypropyl ethylenediamine, or arginine Ensulizole. These salts protect Ensulizole from crystallization. The statement that Ensulizole is used as a salt, in particular as a salt of an amine base compound, in particular as tromethamine Ensulizole, triethanolamine Ensulizole, tetrahydroxypropyl ethylenediamine, or arginine Ensulizole means that the Ensulizole is present in form of a dissolved salt in the sunscreen or daily care composition. This can be achieved either by providing the salt form of Ensulizole prior to adding it to the sunscreen or daily care composition or in-situ in the sunscreen or daily care composition by providing the neutralizing compound, in particular tromethamine, triethanolamine, tetrahydroxypropyl ethylenediamine, or arginine to the sunscreen or daily care composition separately.

According to one preferred embodiment of the invention, the sunscreen or daily care composition comprises
(i) bis-ethylhexyloxyphenol methoxyphenyl triazine (bemotrizinol) in an amount of 0.5 to 6.0 wt. %;
(ii) 1-(4-(1,1-dimethylethyl)phenyl)-3-(4-methoxyphenyl)propane-1,3-dione (avobenzone) in an amount of 1.0 to 5.0 wt. %;
(iii) a water-soluble organic UV filter in an amount of 1.0 to 4.0 wt. %;
(iv) butyloctyl salicylate in an amount of 0.5 to 20.0 wt. %;
each based on the total weight of the sunscreen or daily care composition.

According to an even more preferred embodiment of the invention, the sunscreen or daily care composition comprises
(i) bis-ethylhexyloxyphenol methoxyphenyl triazine (bemotrizinol) in an amount of 2.5 to 6.0 wt. %;
(ii) 1-(4-(1,1-dimethylethyl)phenyl)-3-(4-methoxyphenyl)propane-1,3-dione (avobenzone) in an amount of 2.5 to 5.0 wt. %;
(iii) a water-soluble organic UV filter in an amount of 1.0 to 3.0 wt. %;
(iv) butyloctyl salicylate in an amount of 2.0 to 15.0 wt. %;
each based on the total weight of the sunscreen or daily care composition.

Other advantageous amounts of bis-ethylhexyloxyphenol methoxyphenyl triazine (bemotrizinol) in the inventive sunscreen or daily care composition are 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, each based on the total weight of the sunscreen or daily care composition as well as any values in between, for example 2.5 wt. % to 6.0 wt. %., 3.0 wt. % to 5.5 wt. %, 3.5 wt. % to 5.0 wt. %, 4.0 to 4.5 wt. %, each based on the total weight of the sunscreen or daily care composition. Other advantageous amounts of 1-(4-(1,1-dimethylethyl)phenyl)-3-(4-methoxyphenyl)propane-1,3-dione (avobenzone) in the inventive sunscreen or daily care composition are 2.5 wt. %, 3.0 wt. %, 3.5 wt. %, 4.0 wt. %, 4.5 wt. %, 5.0 wt. %, each based on the total weight of the sunscreen or daily care composition as well as any values in between, for example 2.5 wt. % to 5.0 wt. %, 3.0 wt. % to 4.5 wt. %, 3.5 wt. % to 4.0 wt. %, each based on the total weight of the sunscreen or daily care composition. Other advantageous amounts of the water-soluble organic UV filter (iii) UV filter are 1.5 wt. %, 2.0 wt. %, or 2.5 wt. %, each based on the total weight of the sunscreen or daily care composition.

According to a preferred embodiment of the invention, the inventive sunscreen or daily care composition exhibits an in vitro water resistance of at least 41%, in particular of at least 45% or of at least 46% or of at least 47% or of at least 48% or of at least 49% or of at least 50% or of at least 51% or of at least 52% or of at least 53% or of at least 54% or of at least 55% or of at least 56% or of at least 57% or of at least 58%. The term “water resistance” was determined from spectroscopic measurements and is based on the “solution method” in Sohn et al., Int J Cosmet Sci, “In vitro water resistance testing using SPF simulation based on spectroscopic analysis of rinsed sunscreens”, 2018; DOI:10.1111/ics.12455. The steps of sunscreens application and water immersion plus rinsing and spectroscopic measurements are the same as described in Sohn et al. The sunscreen to be studied was applied on substrate plates made of ethylene methacrylate acid copolymer (described in Sohn et al., Skin Pharmacol Physiol, “Development of a Synthetic Substrate for the in vitro Performance Testing of Sunscreens”, 2017, 30:159-170) at an amount of 2 mg/cm². Two sunscreen-covered plates were immersed in a water bath and two were kept at ambient temperature in dark. After immersion, each of the four plates (two subjected and two not subjected to water immersion) was rinsed with a solvent mixture composed of THF/Ethanol/tetrahydroxypropyl ethylenediamine (Neutrol TE) (50:48:2) to recover the UV filters remained on the plates. The solvent/formulation solution was diluted (1:40 dilution) and filled into a 1 cm quartz cuvette for UV spectroscopic measurements from 290 to 400 nm in 5 nm step using a Lambda 20 device. The result is obtained as transmittance data, converted into absorbance values, from which the area under the curve was calculated. The area under the curve is then used for the calculation of the percentage of the water resistance (% WR).

A water resistance of 30% for example means that for a composition retains 30% of its original performance in terms of absorbance over the full UV range. Considering the absorbance spectrum from 290 to 400 nm (and not for example the SPF value as in Sohn et al., Skin Pharmacol Physiol, 2017, 30:159-170) ensures that the full UV range UVB plus UVA is considered for the water resistance efficacy of the composition. Further details regarding the preferred method to determine the water resistance are described in the experimental section.

In another embodiment of the invention, the sunscreen or daily care composition has a SPF of more than 15 or more than 30 or more than 50 or more than 60. The SPF value can be determined by using a SPF calculation system for in-silico determination of the SPF, for example using the BASF sunscreen simulator (https://sunscreensimulator.basf.com/). The calculation method is further described in B. Herzog et al. Simulation of sunscreen performance, Pure Appl. Chem. 2015; 87(9-10):937-951. Alternatively, the SPF can be determined in-vivo (according to ISO 24444) or in-vitro. SPF in vitro determination is based on the assessment of UV-transmittance through a thin film of sunscreen sample spread on roughened substrate which is transparent to UV. Suitable substrates are for example PMMA plates (an example of PMMA plates is the SB6 PMMA-plates (roughness Ra is around 4-5 μm, area 4.8×4.8 cm) from Helioscreen, Creil, France). The sunscreen formulation to be tested is applied on the PMMA plate with an amount of 1.2 mg/cm² for a plate with a roughness of Ra of 4-5 μm. This amount may differ if a plate with different roughness is used as substrate. Three plates and 5 measurements per plate are done per formulation. The formulation is distributed manually as equally as possible on the plate. The plates applied with formulation are then stored 30 minutes at room temperature (22° C.) and in a dark place prior the measurement.

In vitro transmission measurement may be performed from 290-400 nm with 1 nm steps with the Labsphere UV Transmittance Analyzer UV 2000S. The UV transmission spectrum is then acquired, and the SPF in vitro values are calculated according to equation 1 (mean SPF of all 15 values (from the three plates and 5 measurements per plate))

$$SRF = \frac{{\sum }_{290 nm}^{400 nm} {s}_{er} ( λ ) \cdot S ( λ )}{{\sum }_{290 nm}^{400 nm} {s}_{er} ( λ ) \cdot S ( λ ) \cdot T ( λ )} \qquad Eq . ( 1 )$$

where, sₑᵣ(λ) is the erythema action spectrum and S(λ) is the spectral irradiance received from the UV source and T(λ) is the in vitro measured light transmittance.

It is further preferred that the sunscreen or daily care composition has a protection ratio UVA1/UV equal or superior to 0.7 (according to the FDA proposed order (OTC000008) issued Sep. 24, 2021).

It is further preferred that the sunscreen or daily care composition has a ratio UVA-PF/SPF superior or equal to ⅓ (according to the EC recommendation of 22 Sep. 2006 on the efficacy of sunscreen products and the claims made relating thereto), more preferred of more than 0.4. The UVA-PF can be measured in vivo (ISO24442), in vitro (ISO24443) or also be calculated in-silico, preferably using the BASF sunscreen simulator (https://sunscreensimulator.basf.com/).

In a preferred embodiment of the present invention, the sunscreen or daily care composition provides a photostability of more than 80%, preferably of more than 85%, and in particular of more than 90%. Preferably, the sunscreen or daily care composition provides a photostability after 10 MED, 20 MED, and/or 50 MED, of more than 80%, preferably of more than 85%, and in particular of more than 90%. The photostability of the compositions is preferably determined by applying the respective composition on roughened quartz plates (2 μl/cm²) and then irradiating the plates using an Atlas CPS device with a certain minimal erythemal dose (MED) irradiation, preferably 50 MED irradiation. Then, the plates are rinsed off with tetrahydrofuran and the rinsing solution is analyzed via HPLC to determine the recovery of the UV filters. An alternative preferred method for determining the photostability of the composition comprises the irradiation of sunscreen-covered PMMA plates and evaluation of the difference between the absorbance spectrum before and after irradiation.

The inventive sunscreen and daily care compositions preferably comprise additives, in particular selected from the group consisting of emulsifiers, emollients, further viscosity regulators (thickeners), sensory enhancers, adjuvants, preservatives, perfumes and combinations thereof.

Preferred O/W emulsifiers include
glucose derivatives such as cetearyl glucoside, arachidyl glucoside, lauryl glucoside, polyglyceryl-3 methylglucose distearate, methyl glucose sesquistearate;
sucrose derivative such as sucrose polystearate, sucrose palmitate;
sorbitol derivatives such as polysorbate derivatives;
inulin derivatives such as inulin lauryl carbamate;
glycerides of fatty acids such as glyceryl stearate, glyceryl stearate SE, glyceryl stearate citrate, glyceryl oleate;
glumatic acid derivatives such as sodium stearoyl glutamate;
sulfosuccinic acid derivatives such as disodium cetearyl sulfosuccinate;
phosphoric acid derivatives such as potassium cetyl phosphate, ceteh-10 phosphate, C20-22 alkyl phopshate;
fatty acid esters of polyglyceryl such as polyglyceryl-3-diisostearate, polyglyceryl-2-dipolyhydroxystearate; polyglyceryl-10 stearate
oxyalkenylated fatty alcohol such as ceteareth-20, steareth-21, beneheth-25
oxyalkenylated fatty acid such as PEG-100 stearate
oxyalkenylated organomodified silicone/polysiloxane/polyalkyl/polyether copolymers and derivatives.
phospholipids based such as lecithin derivatives

Preferred emulsifiers to form a W/O emulsion include
glycerides of fatty acids such glyceryl oleate, sorbitan laurate
sorbitan esters such as sorbitan oleate
fatty acid esters of polyglyceryl such as polyglyceryl-3-diisostearate, polyglyceryl-2-dipolyhydroxystearate, polyglyceryl-4 isostearate
Oxyakenylated fatty alcohol such steareth-2
Oxyakenylated fatty acid such as PEG-30 dipolyhydroxystearate
organomodified silicone/polysiloxane/polyalkyl/polyether copolymers and derivatives such as cetyl dimethicone copolyol, cetyl PEG/PPG-10/1 Dimethicone, PEG-10 dimethicone

Preferred emollients include
esters of linear or branched fatty acids with linear or branched fatty alcohols such as propylheptyl caprylate, coco caprylate, isopropyl myristate, ethylhexyl palmitate;
esters of aromatic carboxylic acids with linear or branched fatty alcohols such as C12-C15-alkyl benzoate, ethylhexyl benzoate, phenethyl benzoate;
dicarboxylic acid esters with linear or branched alcohols such as dibutyl adipate, dicaprylyl carbonate, diisopropyl sebacate;
esters of hydroxycarboxylic acids with linear or branched fatty alcohols;
esters of linear or branched fatty acids with polyhydric alcohol such as butylene glycol dicaprylate/dicaprate;
mono-, di-, tri-glycerides based on C6-C18 fatty acids such as caprylic/capric triglycerides, coco glycerides;
guerbet alcohols such as octyldodecynol;
hydrocarbons such as hydrogenated polyisobutene, mineral oil, squalene, isohexadecane;
ethers such as dicaprylyl ether;
silicone derivatives (organomodified polysiloxanes) such as dimethylpolysiloxane, cyclic silicones.
hydrocarbons such as isododecane, isohexadecane

Preferred lipophilic thickeners include
fatty alcohols such as cetyl alcohol, cetearyl alcohol, stearyl alcohol; behenyl alcohol
fatty acids such as stearic acid; palmitic acid
fatty acid esters such as myristyl stearate, pentaerythrityl distearate, cetyl palmitate; Tribehenin, dextrin palmitate
waxes such as beeswax, carnauba wax, microcrystalline wax, ceresin, ozocerite; Oryza sativa bran Wax, sunflower wax
hydrogenated vegetable oil, hydrogenated castor oil, hydrogenated vegetable glycerides,
hydrogenated castor oil/sebacic acid copolymer
poly C10-30 alkyl acrylate
polyamide derivatives such as polyaminde-8
silica based derivatives such as silica, silica dimethyl silylate

### Hydrophilic Stabilizers/Thickeners

natural gums such as xanthan gum, tara gum, carrageenan
silicate derivatives such as magnesium aluminium silicates;
cellulose derivatives such as hydroxypropyl cellulose, microcrystalline cellulose
polyacrylic acid based derivatives such as sodium polyacrylate, Acrylates/C10-30 Alkyl Acrylate Crosspolymer, carbomer, Acrylates/Beheneth-25 Methacrylate Copolymer, Hydroxyethyl Acrylate/Sodium Acryloyldimethyl Taurate Copolymer
starch derivatives such as hydroxypropyl starch phosphate

### UV Protection Scattering Compound

Styrene/Acrylates Copolymer
Microcrystalline cellulose
Calcium carbonate
Hydroxyapatite
silica

### Film Formers/Film Forming Enhancers

Polyvinylpyrrolidone based compounds such as Triacontanyl PVP, VP/hexadecane copolymer, VP/Eicosene Copolymer, hydrolyzed wheat protein/PVP crosspolymer
Hydrogenated Dimer Dilinoleyl/Dimethylcarbonate Copolymer
Diisostearoyl Polyglyceryl-3 Dimer Dilinoleate
Polyglyceryl-3 Stearate/Sebacate Crosspolymer
Polyacrylate-15
Oryza sativa bran Wax
Carnauba wax
Cellulose derivatives such as Methylcellulose, ethylcellulose
Hydrogenated Castor oil
Capryloyl Glycerin/Sebacic Acid Copolymer
Acrylates/Octylacrylamide Copolymer

Preferred sensory enhancers include
silica;
mica;
polymethylsilsesquioxane;
starch derivatives such as aluminum starch octenylsuccinate; rice starch
dimethicone derivatives;
boron nitride;
HDI/trimethylol hexyllactone crosspolymer.
perlite

Preferred humectants include
glycerin
Butylene glycol
Propylene glycol
beta-glucan
1,2 pentadiol
1,2 hexandiol
1,2 octandiol
1,2 decandiol
2-Methyl-1,3-propandiol.

Preferred adjuvants include
tocopherol derivatives;
retinol derivatives;
ascorbic acid derivatives;
bisabolol;
allantoin;
panthenol;
chelating agents (EDTA, EDDS, EGTA, phytic acid, piroctone olamine);
ethylhexyl glycerin;
hydroxyacetophenone;
caprylhydroxymic acid;
propellants such as propane, butane, isobutene, dimethyl ether;
insect repellants such as butylacetylaminopropionate.

Preferred preservatives include the compounds listed in annex V of the Regulation 1223/2009/EC on Cosmetic Products, in particular
benzyl alcohol;
zingerone.

Preferred perfumes are selected from the group consisting of limonene, citral, linalool, alpha-isomethylionon, geraniol, citronellol, 2-isobutyl-4-hydroxy-4-methyltetrahydropyrane, 2-tert.-pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl C butylphenylmehtylpropionalcinnamal, amylsalicylate, amylcinnamylalcohol, anisalcohol, benzoin, benzylalcohol, benzylbenzoate, benzylcinnamate, benzylsalicylate, bergamot oil, bitter orange oil, butylphenylmethylpropioal, cardamom oil, cedrol, cinnamal, cinnamylalcohol, citronnellylmethylcrotonate, lemon oil, coumarin, diethylsuccinate, ethyllinalool, eugenol, evernia furfuracea extracte, evernia prunastri extracte, farensol, guajak wood oil, hexylcinnamal, hexylcalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, vanillin and combinations thereof.

It is preferably that the inventive sunscreen or daily care composition comprises two or more of the additives described above. In connection with the above preferred embodiments, it is to be understood that if the sunscreen or daily care composition comprises two or more additives, combinations of any of the additives as defined above are also part of the invention.

In connection with the above preferred and particularly preferred embodiments, it is to be understood that the sunscreen or daily care composition may further comprise water.

In case water is present in the sunscreen or daily care composition it is to be understood that it is preferably present in an amount of more than 5% by weight, based on the total weight of the composition. Further, it is to be understood that in case water is present in the sunscreen or daily care composition, the sunscreen or daily care composition can be an oil in water emulsion (O/W emulsion), a water in oil emulsion (W/O emulsion), a gel cream or an oil in gel.

In one embodiment of the present invention, the sunscreen or daily care composition can be provided in different forms, e.g. gels, creams, oils, lotions, sticks, or in the form of a sprayable product.

In a second aspect, the invention relates to a method for increasing the water resistance of a sunscreen or daily care composition comprising the step of adding a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, Bis(Cyano Butylacetate) Anthracenediylidene, polyester-25, butyloctyl salicylate and combinations thereof to a sunscreen or daily care composition comprising (i) bis-ethylhexyloxyphenol methoxyphenyl triazine (bemotrizinol), (ii) 1-(4-(1,1-dimethylethyl)phenyl)-3-(4-methoxyphenyl)propane-1,3-dione (avobenzone), and (iii) a water-soluble organic UV filter. It was discovered that the addition of a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, Bis(Cyano Butylacetate) Anthracenediylidene, polyester-25, butyloctyl salicylate and combinations thereof to a sunscreen or daily care composition comprising (i) bemotrizinol, (ii) avobenzone, and (iii) a water-soluble, organic UV absorber results in an increased water resistance of the composition.

All the statements made regarding the components and properties of the sunscreen or daily care composition with regard to the first aspect of the invention also apply mutatis mutandis to the second aspect of the invention.

The invention further relates to a sunscreen or daily care composition comprising a UV filter combination of
(i) bis-ethylhexyloxyphenol methoxyphenyl triazine (bemotrizinol) and
(ii) 1-(4-(1,1-dimethylethyl)phenyl)-3-(4-methoxyphenyl)propane-1,3-dione (avobenzone)
(iii) Phenyl-3H-benzimidazole-5-sulfonic acid (Ensulizole); and further comprising
(iv) a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, Bis(Cyano Butylacetate) Anthracenediylidene, polyester-25, butyloctyl salicylate and combinations thereof; and being free of any further UV filter.

The invention is further illustrated by the following examples.

### EXAMPLES

### Methods

### Water Resistance In Vitro Solution Method

The in vitro water resistance was determined from spectroscopic measurements and is based on the “solution method” in Sohn et al., Int J Cosmet Sci, “In vitro water resistance testing using SPF simulation based on spectroscopic analysis of rinsed sunscreens”, 2018; DOI:10.1111/ics.12455. The steps of sunscreens application and water immersion plus rinsing and spectroscopic measurements are the same as described in Sohn et al.

### Sunscreen Application:

The sunscreen to be studied was applied on substrate plates made of ethylene methacrylate acid copolymer having the dimension of 5 cm×5 cm (described in Sohn et al., Skin Pharmacol Physiol, “Development of a Synthetic Substrate for the in vitro Performance Testing of Sunscreens”, 2017, 30:159-170; DOI: 10.1159/000464471). These conditions are understood to be the current most adequate approach for determining the in vitro water resistance of formulations.

On each plate an amount of 2 mg/cm2 (corresponding to 50 mg of the formulation per plate) was applied. In total, four plates were prepared per sunscreen. After sunscreen application followed by an equilibration period of 15 min, two of the four plates were immersed in a water bath and the two remaining plates were kept at ambient temperature in dark.

### Water Immersion Condition

The sunscreen-covered plates were attached using a system of strips from Tesa Hook & Loop tape at a defined height on the edges of a 1000-mL beaker (diameter of 10 cm). The beaker was filled with 500 mL of distilled water with controlled temperature of 30° C.±2° C. The water was stirred with a speed of 300 rpm for 20 min using a three-bladed propeller stirrer in an equidistant position from the attached plates. Afterwards, the plates were removed from the emptied beaker and were allowed to dry at ambient temperature for 30 min before continuation.

### Spectroscopic Measurements and Determination of the Water Resistance Value

After immersion, each of the four plates (two subjected and two not subjected to water immersion) was put in a separate beaker and washed using 20 mL of a solvent mixture composed of THF/ethanol/tetrahydroxypropyl ethylenediamine (neutrol TE) (50:48:2). To collect the sunscreen from the plate, each plate was rinsed 10 times using the same solvent with a Pasteur pipette holding the plate out of the solvent with tweezers to remove visually the entire sunscreen residue from the substrate plate. The solvent/formulation solution was diluted (1:40 dilution) and filled into a 1-cm quartz cuvette for UV spectroscopic measurements from 290 to 400 nm in 5 nm step using a Lambda 20 device. The water resistance retention was deduced from UV transmittance measurement of the respective sunscreen solution resulting from the washing of the substrate plate with and without water immersion. The transmittance data are then converted into absorbance data according to equation 2:

$$A ( λ ) = - log T ( λ ) \qquad Eq . ( 2 )$$

wherein A(λ) us the absorbance and T(λ) the transmittance at wavelength A.

The area under the curve (AUC) for the two plates subjected (AUC_{wet}) and not subjected to water immersion (AUC_{static}) was calculated from the absorbance data and used for the calculation of the percentage of the water resistance (% WR),

As different plates are used to determine the AUC_{wet} and AUC_{static}, the % WR is calculated from the ratio between the average wet and average static as described in the equation 3 below:

$$\% WR = \frac{{AUC}_{wet}}{{AUC}_{static}} \cdot 100 , \qquad Eq . ( 3 )$$

wherein AUC_{wet} is the average of the area under the curve for the two plates subjected to water immersion and AUC_{dry} is the average of the area under the curve for the two plates not subjected to water immersion (dry).

### Formulations

TABLE 1
Oil/Water formulations

| | | Inv. | Comp. |
|---|---|---|---|
| | | formulation | formulation |
| Part | INCI | 1 [wt. %] | 2 [wt. %] |
| A | Disodium Cetearyl Sulfosuccinate | 1.00 | 1.00 |
| | Cetearyl Alcohol | 2.00 | 2.00 |
| | C12-15 Alkyl benzoate | 10.00 | 10.00 |
| | Dibutyl Adipate | 10.00 | 10.00 |
| | Dicaprylyl Carbonate | 8.00 | 8.00 |
| | Phenoxyethanol, Ethylhexylglycerin | 1.00 | 1.00 |
| | Butyloctyl Salicylate | 6.00 | — |
| | Butyl Methoxydibenzoylmethane | 4.00 | 4.00 |
| | (avobenzone) | | |
| | Bis-Ethylhexyloxyphenol Methoxy- | 5.00 | 5.00 |
| | phenyl Triazine (bemotrizinol) | | |
| B | Water | q.s. to 100 | q.s. to 100 |
| | Glycerin | 2.00 | 2.00 |
| | Sodium Polyacrylate | 0.30 | 0.30 |
| | Xanthan Gum | 0.20 | 0.20 |
| | Disodium EDTA | 0.20 | 0.20 |
| C | Phenylbenzimidazole Sulfonic Acid | 2.50 | 2.50 |
| | (Ensulizole) | | |
| | Water | 10.00 | 10.00 |
| | Tromethamine | 2.00 | 2.00 |
| | Water resistance (in vitro | 58% | 40% |
| | measurement; % WR) | | |

The compositions inventive formulation 1 and comparative formulation 2 were manufactured as follows:

Part A was heated to 80° C. while stirring until the mixture was fully homogenous.

Part B was heated to 80° C. while stirring. The ingredients of part C were mixed. Then, part A was added to part B while stirring. Then, part C previously prepared was added to the mixture of parts A and B. The combined mixture was then homogenized using an ultra turrax device. The formulation was cooled down to room temperature (22° C.) under stirring.

SPF, UVA-PF/SPF, and UVA1/UV ratio values for the filter system used in the formulations were calculated using in-silico determination methods using the BASF sunscreen simulator (https://sunscreensimulator.basf.com/). The SPF in silico was 28, the UVA-PF/SPF was 0.56 and the UVA1/UV ratio was 0.79. The UVA1/UV ratio was calculated using the final extinction spectrum available in an excel file using the export function of the calculation.

The two compositions were compared regarding their water resistance. The water resistance of the comparative formulation 2 was only 40%. The addition of butyloctyl salicylate led to a substantial increase of the water resistance for inventive formulation 1 up to 58%, which is an increase of almost half over the value obtained for the comparative formulation 2.

1. Sunscreen or daily care composition comprising
(i) bis-ethylhexyloxyphenol methoxyphenyl triazine (bemotrizinol),
(ii) 1-(4-(1,1-dimethylethyl)phenyl)-3-(4-methoxyphenyl)propane-1,3-dione (avobenzone),
(iii) a water-soluble organic UV filter,
(iv) a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, bis(cyano butylacetate) anthracenediylidene, polyester-25, butyloctyl salicylate and combinations thereof.

2. The sunscreen or daily care composition according to claim 1,
wherein the sunscreen or daily care composition is free of the UV filters from the group consisting of 4,4′,4″-(1,3,5-triazine-2,4,6-triyltriimino)tris-benzoic acid-tris(2-ethylhexyl)ester (octyl triazone), hexyl 2-[4-(diethylamino)-2-hydroxybenzoyl]benzoate (INCI diethylamino hydroxybenzoyl hexyl benzoate), 2,2′-Methylenbis[6-(2H-benzotriazol-2-yl)-4-(1,1,3,3-tetramethylbutyl)phenol](bisoctrizole), 3,3,5-Trimethylcyclohexyl 2-hydroxybenzoate (INCI Homosalate), (RS)-2-Ethylhexyl (2E)-3-(4-methoxyphenyl)prop-2-enoate (INCI ethylhexyl methoxycinnamate), and 2-hydroxy-4-methoxybenzophenone (INCI benzophenone-3).

3. The sunscreen or daily care composition according to claim 1, wherein the sunscreen is free of 2-ethylhexyl-2-cyano-3,3-diphenylprop-2-enoate (INCI octocrylene).

4. Sunscreen or daily care composition according to claim 1, wherein the sunscreen or daily care composition is free of any further organic UV filter.

5. Sunscreen or daily care composition according to claim 1, wherein the sunscreen or daily care composition is free of zinc oxide.

6. Sunscreen or daily care composition according to claim 1, wherein the sunscreen or daily care composition is free of titanium oxide.

7. Sunscreen or daily care composition according to claim 1, wherein the sunscreen or daily care composition is free of any inorganic UV filter.

8. Sunscreen or daily care composition according to claim 1, wherein the weight ratio of (i) bis-ethylhexyloxyphenol methoxyphenyl triazine (bemotrizinol) to (ii) 1-(4-(1,1-dimethylethyl)phenyl)-3-(4-methoxyphenyl)propane-1,3-dione (avobenzone) is from 1:3 to 3:1.

9. Sunscreen or daily care composition according to claim 1, wherein the water-soluble organic UV filter (iii) is selected from the group consisting of Phenyl-3H-benzimidazole-5-sulfonic acid (Ensulizole), [(3Z)-3-[[4-[(Z)-[7,7-Dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (Ecamsule), disodium phenyl dibenzimidazole tetrasulfonate (Bisdisulizole disodium), and mixtures thereof.

10. Sunscreen or daily care composition according to claim 9, wherein the water-soluble organic UV filter (iii) is Phenyl-3H-benzimidazole-5-sulfonic acid (Ensulizole), wherein the sunscreen or daily-care composition further comprises a neutralizing agent selected from tromethamine, triethanolamine, tetrahydroxypropyl ethylenediamine, and arginine.

11. Sunscreen or daily care composition according to claim 1, wherein the sunscreen or daily care composition comprises
(i) bis-ethylhexyloxyphenol methoxyphenyl triazine (bemotrizinol) in an amount of 0.5 to 6.0 wt. %;
(ii) 1-(4-(1,1-dimethylethyl)phenyl)-3-(4-methoxyphenyl)propane-1,3-dione (avobenzone) in an amount of 1.0 to 5.0 wt. %;
(iii) a water-soluble organic UV filter in an amount of 1.0 to 4.0 wt. %;
(iv) butyloctyl salicylate in an amount of 0.5 to 20.0 wt. %;
each based on the total weight of the sunscreen or daily care composition.

12. Sunscreen or daily care composition according to claim 1, wherein the sunscreen or daily care composition comprises
(i) bis-ethylhexyloxyphenol methoxyphenyl triazine (bemotrizinol) in an amount of 2.5 to 6.0 wt. %;
(ii) 1-(4-(1,1-dimethylethyl)phenyl)-3-(4-methoxyphenyl)propane-1,3-dione (avobenzone) in an amount of 2.5 to 5.0 wt. %;
(iii) a water-soluble organic UV filter in an amount of 1.0 to 3.0 wt. %;
(iv) butyloctyl salicylate in an amount of 2.0 to 15.0 wt. %;
each based on the total weight of the sunscreen or daily care composition.

13. Sunscreen or daily care composition according to claim 1, wherein the sunscreen or daily care composition exhibits an in vitro water resistance of at least 50%.

14. Sunscreen or daily care composition according to claim 1, wherein the sunscreen or daily care composition has a SPF of more than 15 or more than 30 or more than 50 and/or wherein the sunscreen or daily care composition has a photostability of more than 80%.

15. Method for increasing the water resistance of a sunscreen or daily care composition comprising the step of adding a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, Bis(Cyano Butylacetate) Anthracenediylidene, polyester-25, butyloctyl salicylate and combinations thereof to a sunscreen or daily care composition comprising (i) bis-ethylhexyloxyphenol methoxyphenyl triazine (bemotrizinol), (ii) 1-(4-(1,1-dimethylethyl)phenyl)-3-(4-methoxyphenyl)propane-1,3-dione (avobenzone), and (iii) a water-soluble organic UV filter.

16. Sunscreen or daily care composition comprising a UV filter combination of
(i) bis-ethylhexyloxyphenol methoxyphenyl triazine (bemotrizinol) and
(ii) 1-(4-(1,1-dimethylethyl)phenyl)-3-(4-methoxyphenyl)propane-1,3-dione (avobenzone)
(iii) Phenyl-3H-benzimidazole-5-sulfonic acid (Ensulizole); and further comprising
(iv) a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, Bis(Cyano Butylacetate) Anthracenediylidene, polyester-25, butyloctyl salicylate and combinations thereof; and being free of any further UV filter.

17. Sunscreen or daily care composition comprising a UV filter combination of
(i) bis-ethylhexyloxyphenol methoxyphenyl triazine (bemotrizinol) and
(ii) 1-(4-(1,1-dimethylethyl)phenyl)-3-(4-methoxyphenyl)propane-1,3-dione (avobenzone)
(iii) Phenyl-3H-benzimidazole-5-sulfonic acid (Ensulizole); further comprising
(iv) a compound selected from the group consisting of benzotriazolyl dodecyl p cresol, ethylhexyl methoxycrylene, polyester-8, diethylhexyl syringylidenemalonate, trimethoxybenzylidene pentanedione, diethylhexyl 2,6-naphthalate, Bis(Cyano Butylacetate) Anthracenediylidene, polyester-25, butyloctyl salicylate and combinations thereof;
and being free of any further UV filter;
wherein the sunscreen or daily care composition comprises a compound selected from the group consisting of tromethamine, triethanolamine, tetrahydroxypropyl ethylenediamine, and arginine.
